(19) United States
(12) Patent Application Publication
(10) Pub. No.: US 2013/0296979 A1
(43) Pub. Date: Nov. 7, 2013

(54) COMBINATIONAL HOT COMPRESS DEVICE

(71) Applicant: Ching-Chuan WANG, Keelung (TW)

(72) Inventor: Ching-Chuan WANG, Keelung (TW)

(21) Appl. No.: 13/464,589

(22) Filed: May 4, 2012

(51) Int. Cl. A61F 7/02

(52) U.S. Cl. 607/96

(57) ABSTRACT

A combinational hot compress device comprises a first and second hot compress pads and a first and second positioning elements are respectively disposed around the two hot compress pads. A first and second areas are adjacently disposed on top of the first hot compress pad and a first heating device is disposed in the first area. A third and fourth areas are adjacently disposed on top of the second hot compress pad and a second heating device is disposed in the fourth area. When the first or the second hot compress pad is folded in half, the two halves thereof are coupled by the first or the second positioning element, so that the first and the second areas or the third and the fourth areas are overlapped with each other. Thereby, the two hot compress pads are formed which can be used independently.

### BACKGROUND OF THE INVENTION

1. Field of Invention

The present invention relates to a hot compress device, and more particularly, to a combinational hot compress device which can be used as two independent hot compress pads or as a single hot compress pad assembled from the two hot compress pads according to the need of the user.

2. Related Art

People feel ache and sore in parts of the body because of diseases or bad posture. Many different kinds of hot compress pads can be found in the market for compressing the shoulders, back, abdomen or waist, etc. by heat and relieving the ache and sore.

Generally, the existing hot compress pads have different shapes and are provided in a single piece or two pieces for the hot compress of a single or two parts of the body. Therefore, the user will need to buy two hot compress pads if the hot compress pad is a single piece and the user wants two different parts of the body treated with hot compress. If the hot compress pad is made up of two pieces and the user only needs a single part of the body treated with hot compress, the other additional piece of hot compress pad will make it inconvenient for the application.

In view of the above problems and in order to overcome the drawbacks, a combinational hot compress device is provided which can be combined into a single unit or two units for saving the cost for buying and for convenient application.

### SUMMARY OF THE INVENTION

An object of the present invention is to provide a combinational hot compress device. A first positioning element is used independently for assembling a first hot compress pad and a second positioning element is used independently for assembling a second hot compress pad, or the first hot compress pad and the second hot compress pad are assembled by the first positioning element and the second positioning element into a single unit hot compress pad. Thereby, the combinational hot compress device can be used with flexibility based on the needs of the user and the cost for buying can also be saved.

In order to achieve the above-mentioned object, the combinational hot compress device provided by the present invention comprises a first hot compress pad and a second hot compress pad. A first area and a second area are adjacently disposed on a top surface of the first hot compress pad and a first heating device is disposed in the first area. A first positioning element is disposed around an edge of the first hot compress pad. When the first hot compress pad is folded in half, the overlapped first area and the second area are coupled by the first positioning element and the first heating device is wrapped inside the first hot compress pad. A third area and a fourth area are adjacently disposed on a top surface of the second hot compress pad and a second heating device is disposed in the fourth area. A second positioning element is disposed around an edge of the second hot compress pad. When the second hot compress pad is folded in half, the overlapped third area and the fourth area are coupled by the second positioning element and the second heating device is wrapped inside the second hot compress pad. The second positioning element is corresponded with the first positioning element so that the first and the second hot compress pads can be unfolded independently. When the first area is overlapped with the third area and the second area is overlapped with the fourth area, the first hot compress pad and the second hot compress pad are combined together by the first positioning element and the second positioning element.

In implementation, the first positioning element is a zipper strip disposed around the edge of the first hot compress pad; the second positioning element is a zipper strip disposed around the edge of the second hot compress pad. A front end head of the first positioning element can be connected to a back end of the second positioning element; a back end of the first positioning element can be connected to a front end head of the second positioning element.

In implementation, the combinational hot compress device further comprises a controller connected to an end of the first heating device. A first connector is disposed at another end of the first heating device. A second connector is disposed at an end of the second heating device for connecting with the first connector correspondingly.

In implementation, the combinational hot compress device further comprises a connection wire with a third connector disposed at an end of the connection wire for connecting with the first connector correspondingly. A fourth connector is disposed at another end of the connection wire for connecting with the second connector correspondingly.

In implementation, a first hole is disposed on the first hot compress pad for the first connector to penetrate through, and a second hole is disposed on the second hot compress pad for the second connector to penetrate through.

The present invention will become more fully understood by reference to the following detailed description thereof when read in conjunction with the attached drawings.

### BRIEF DESCRIPTION OF THE DRAWINGS

FIG. 1 is an exploded perspective view showing a first hot compress pad and a second hot compress pad according to a preferred embodiment of the present invention;

FIGS. 2 and 3 are perspective views showing the first hot compress pad and the second hot compress pad being used separately according to the preferred embodiment of the present invention; and

FIG. 4 is a perspective view showing the first hot compress pad and the second hot compress pad being combined together according to the preferred embodiment of the present invention.

### DETAILED DESCRIPTION OF THE INVENTION

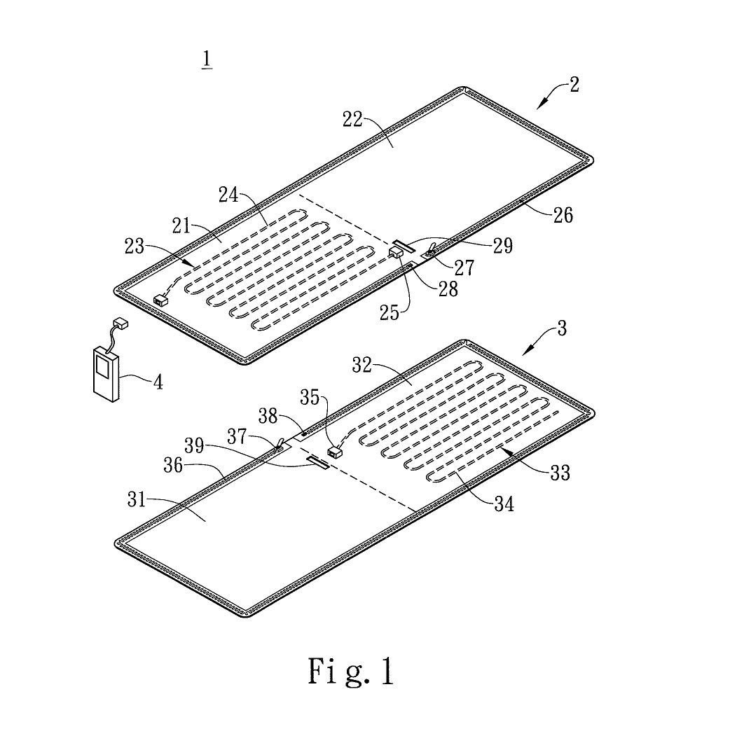

FIG. 1 is an exploded perspective view of a combinational hot compress device 1 according to a preferred embodiment of the present invention. The combinational hot compress device 1 comprises a first hot compress pad 2, a second hot compress pad 3 and a controller 4.

The first hot compress pad 2 is rectangular and its top surface is divided into two areas comprising a first area 21 and a second area 22 adjacently disposed on the top surface. A first heating device 23 is disposed in the first area 21. The first heating device 23 comprises a heating wire 24 being bent many times. The controller 4 is connected to an end of the heating wire 24 for controlling the heating temperature of the heating wire 24. A first connector 25 is disposed at another end of the heating wire 24.

A zipper strip is disposed around an edge of the first hot compress pad 2. The zipper strip comprises a front end head 27 and a back end 28 which can be connected with the front end head 27. The zipper strip is used as a first positioning element 26. A first hole 29 is disposed between the first area 21 and the second area 22 of the first hot compress pad 2. The first hole 29 is also disposed by the first connector 25.

The second hot compress pad 3 and the first hot compress pad 2 are the same in size and shape. A top surface of the second hot compress pad 3 is divided into two areas comprising a third area 31 and a fourth area 32 adjacently disposed on the top surface. A second heating device 33 is disposed in the fourth area 32. The second heating device 33 comprises a heating wire 34 being bent many times. A second connector 35 is disposed at an end of the heating wire 34. The second connector 35 is connected with the first connector 25 correspondingly.

A zipper strip is disposed around an edge of the second hot compress pad 3. The zipper strip comprises a front end head 37 and a back end 38 which can be connected with the front end head 37. The zipper strip is used as a second positioning element 36. A second hole 39 is disposed between the third area 31 and the fourth area 32 of the second hot compress pad 3. The second hole 39 is also disposed by the second connector 35.

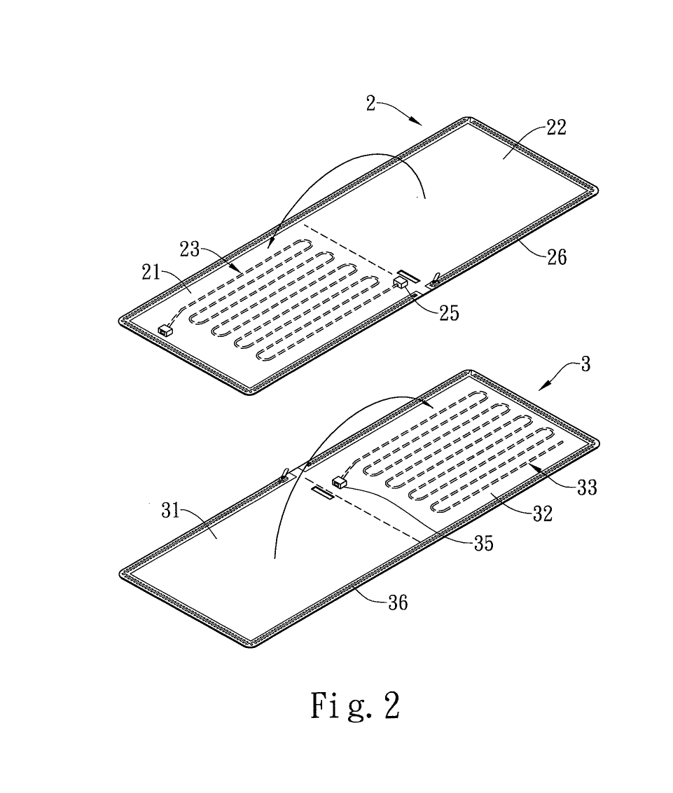

As shown in FIGS. 2 and 3, when the first hot compress pad 2 is folded in half, the overlapped first area 21 and the second area 22 are coupled by the first positioning element 26 and the first heating device 23 is wrapped inside the first hot compress pad 2. When the second hot compress pad 3 is folded in half, the overlapped third area 31 and the fourth area 32 are coupled by the second positioning element 36 and the second heating device 33 is wrapped inside the second hot compress pad 3. When the first connector 25 of the first heating device 23 is connected with the second connector 35 of the second heating device 33 correspondingly, the two hot compress pads are formed which can be used independently for the hot compress of different parts of a human body. As shown in FIG. 3, a connection wire 5 is connected between the first heating device 23 and the second heating device 33. A third connector 51 is disposed at an end of the connection wire 5 and a fourth connector 52 is disposed at another end of the connection wire 5. When the third connector 51 is connected with the first connector 25 correspondingly and the fourth connector 52 is connected with the second connector 35 correspondingly, a distance between the first hot compress pad 2 and the second hot compress pad 3 can be lengthened so that it is more convenient for the user to use the combinational hot compress device 1.

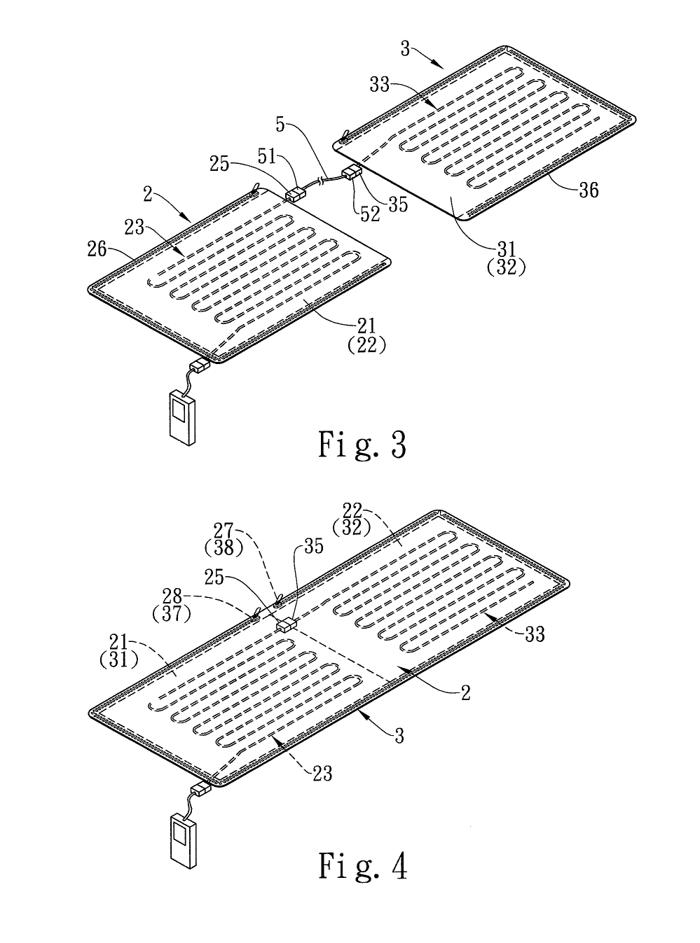

FIG. 4 is another application state of the combinational hot compress device 1 of the present invention. The first hot compress pad 2 and the second hot compress pad 3 are unfolded independently, and the first hot compress pad 2 is disposed on the second hot compress pad 3. The first area 21 of the first hot compress pad 2 is overlapped with the third area 31 of the second hot compress pad 3. The second area 22 of the first hot compress pad 2 is overlapped with the fourth area 32 of the second hot compress pad 3. Thereby, the first heating device 23 is interposed between the first area 21 and the third area 31; the second heating device 33 is interposed between the second area 22 and the fourth area 32. When the first connector 25 of the first hot compress pad 2 is connected with the second connector 35 of the second hot compress pad 3, the front end head 27 of the first positioning element 26 is coupled with the back end 38 of the second positioning element 36 correspondingly, and the back end 28 of the first positioning element 26 is coupled with the front end head 37 of the second positioning element 36 correspondingly, the first hot compress pad 2 and the second hot compress pad 3 are combined into one unit for large area application on the user.

In implementation, the first positioning element 26 and the second positioning element 36 can also be Velcro strips for assembling the first hot compress pad 2 and the second hot compress pad 3 independently. The first hot compress pad 2 and the second hot compress pad 3 can also be combined as a single unit. Therefore, the combinational hot compress device 1 can be used with flexibility based on the needs of the user and the cost for buying can also be saved.

As mentioned from the above, the object of the present invention can be achieved by the simple structured combinational hot compress device which can be separated into two hot compress pads for independent application or can be combined into a single hot compress pad unit for large area application. Thereby, the combinational hot compress device is convenient for usage and the cost for buying can be saved.

Although the embodiments of the present invention have been described in detail, many modifications and variations may be made by those skilled in the art from the teachings disclosed hereinabove. Therefore, it should be understood that any modification and variation equivalent to the spirit of the present invention be regarded to fall into the scope defined by the appended claims.

What is claimed is:

1. A combinational hot compress device, comprising:
a first hot compress pad, having a first area and a second area adjacently disposed on a top surface thereof, where a first heating device is disposed in the first area, and a first positioning element is disposed around an edge of the first hot compress pad for coupling the overlapped first area and the second area and wrapping the first heating device inside the first hot compress pad when the first hot compress pad is folded in half; and
a second hot compress pad, having a third area and a fourth area adjacently disposed on a top surface thereof, where a second heating device is disposed in the fourth area, and a second positioning element is disposed around an edge of the second hot compress pad for coupling the overlapped third area and the fourth area and wrapping the second heating device inside the second hot compress pad when the second hot compress pad is folded in half; the second positioning element is corresponded with the first positioning element so that the first and the second hot compress pads are unfolded independently; and when the first area is overlapped with the third area and the second area is overlapped with the fourth area, the first hot compress pad and the second hot compress pad are combined together by the first positioning element and the second positioning element.

2. The combinational hot compress device as claimed in claim 1, wherein the first positioning element is a zipper strip disposed around the edge of the first hot compress pad, and the second positioning element is a zipper strip disposed around the edge of the second hot compress pad; a front end head of the first positioning element is connectable to a back end of the second positioning element, and a back end of the first positioning element is connectable to a front end head of the second positioning element.

3. The combinational hot compress device as claimed in claim 2, further comprising a controller connected to an end of the first heating device, wherein a first connector is disposed at another end of the first heating device while a second connector is disposed at an end of the second heating device for connecting with the first connector correspondingly.

4. The combinational hot compress device as claimed in claim 3, further comprising a connection wire that is provided with a third connector at one end thereof for connecting the first connector correspondingly and with a fourth connector disposed at another end thereof for connecting the second connector correspondingly.

5. The combinational hot compress device as claimed in claim 4, wherein the first hot compress pad includes a first hole for the first connector to go through and the second hot compress pad has a second hole for the second connector to go through.

6. The combinational hot compress device as claimed in claim 1, further comprising a controller connected to an end of the first heating device, wherein a first connector is disposed at another end of the first heating device while a second connector is disposed at an end of the second heating device for connecting with the first connector correspondingly.

7. The combinational hot compress device as claimed in claim 6, further comprising a connection wire that is provided with a third connector at one end thereof for connecting the first connector correspondingly and with a fourth connector disposed at another end thereof for connecting the second connector correspondingly.

8. The combinational hot compress device as claimed in claim 7, wherein the first hot compress pad includes a first hole for the first connector to go through and the second hot compress pad has a second hole for the second connector to go through.
